SEQUENCE LISTING (4 sequences)

SEQ ID NO: 1 - protein, 235 aa, Artificial Sequence
  Uri22 Kappa amino acid sequence
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 467 aa, Artificial Sequence
  Uri22 Gamma amino acid sequence
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 708 bp, Artificial Sequence
  Uri22 Kappa nucleic acid sequence
atggaaacccctgcccagctgctgttcctgctgctgctgtggctgcccgacaccaccggcgagatcgtgctgacccagtcccctggcaccctgtccctgagccctggcgagagagccaccctgtcctgcagagcctcccagtccgtgcggggcagatacctggcttggtatcagcagaagcccggccaggcccctcggctgctgatctacggcgcctcttccagagccaccggcatccctgaccggttctccggctctggctccggcaccgacttcaccctgaccatctcccggctggaacccgaggacttcgccgtgttctactgccagcagtacggctcctccccccggacctttggccagggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 4 - DNA, 1404 bp, Artificial Sequence
  Uri22 Gamma nucleic acid sequence
atggaatttggcctgtcctggctgttcctggtggccatcctgaagggcgtgcagtgcgaggtgcagctgctggaatctggcggcggactggtgcagcctggcggctccctgagactgtcttgcgccgcctccggcttcaccttctccagctacgccatgtcctgggtccgacaggcccctggcaagggcctggaatgggtgtccggcatcaccggctccggcggctctacctactacgccgactccgtgaagggccggttcacaatctcccgggacaactccaagaacaccctgtacctgcagatgaactccctgcgcgccgaggacaccgccgtgtactactgtgccaaggaccccggcaccaccgtgatcatgagttggttcgacccctggggccagggcaccctggtcaccgtgtcctctgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

(19) United States
(12) Patent Application Publication
(10) Pub. No.: US 2019/0211055 A1
(43) Pub. Date: Jul. 11, 2019

(54) METHODS OF PURIFYING AND QUALIFYING ANTIBODIES

(71) Applicant: KinBio Ltd., Rechovot (IL)

(72) Inventor: Daphna MIRON, Rehovot (IL)

(21) Appl. No.: 16/332,798

(22) PCT Filed: Sep. 14, 2017
(86) PCT No.: PCT/IL2017/051044

§ 371 Date: Mar. 13, 2019

Related U.S. Application Data
(60) Provisional application No. 62/394,265, filed on Sep. 14, 2016.

(51) Int. Cl. C07K 1/16; C07K 16/28; C07K 1/22; C07K 1/18; G01N 33/68; B01D 15/38; B01D 15/36; B01D 15/18

(52) U.S. Cl. CPC C07K 1/165; C07K 16/2875; C07K 1/22; C07K 1/18; B01D 15/1871; B01D 15/3809; B01D 15/3847; B01D 15/362; G01N 33/6854

(57) ABSTRACT

A method of purifying a recombinant antibody having the complementarity determining regions (CDRs) of denosumab, the method comprising subjecting a preparation comprising said antibody to a mixed mode chromatography on a Capto™ Adhere column in a pH range of 6.2-7.4, thereby purifying the antibody.

### FIELD AND BACKGROUND OF THE INVENTION

The present invention, in some embodiments thereof, relates to methods of purifying and qualifying antibodies.

Denosumab (trade names Prolia® and Xgeva®) is a human monoclonal antibody (mAb) for the treatment of osteoporosis, treatment-induced bone loss, metastases to bone, and giant cell tumor of bone. Denosumab is a RANKL inhibitor which acts by preventing the development of osteoclasts. Denosumab was developed by the biotechnology company Amgen Inc.

Denosumab biosimilars are being developed by many pharmaceutical companies including PanPharmaceuticals USA, Oncobiologics and BioXpress Therapeutics.

A biosimilar product cannot be considered an identical copy of its innovator counterpart. Even very small differences in cell lines or manufacturing processes can have large impacts on potential side effects observed during treatment. Two “similar” biologics could trigger very different immunogenic responses. Thus, substitution of a reference biologic with a biosimilar could have significant clinical consequences. That creates safety concerns among regulators. Regulatory agencies therefore guide biosimilar manufacturers to qualify their products using various biological functional assays.

Therapeutic monoclonal antibodies achieve their effects either directly by inducing apoptosis or indirectly by inducing antibody-dependent, cell-mediated cytotoxicity (ADCC) or complement-dependent cytotoxicity (CDC). The mechanism of action generally involves an antibody's Fab (antigen-binding) and Fc regions. The former binds to antigens; the latter bind to Fc receptors found on monocytes, macrophages, and natural killer cells.

Evaluating a mAb's binding affinity to different types of Fc receptors is important for predicting its therapeutic potential. Specifically, analyzing Fc gamma receptor (Fcγ) binding determines an antibody's ability to recruit immune cells, including natural killer cells: neutrophils and macrophages. Testing mAb binding to Fc gamma receptors is actually a requirement of various regulatory agencies.

BioLayer Interferometry (BLI) and Surface plasma resonance (SPRmeasure antibody binding to recombinant soluble Fc receptors. Flow cytometry measures antibody binding to cells that express certain receptors. Both methods have been formatted as parallel-line assays and demonstrate high levels of accuracy, precision, and linearity. That makes them valuable for use in comparability, potency, and stability assays. These methods also show greater precision and reproducibility than traditional cell-based assays that test for ADCC. Additionally, both are readily able to detect structural (e.g., glycosylation, oxidation, deamidation etc.) differences between two mAbs that can affect molecular function. Both assays are also sensitive to changes in antibody function related to glycosylation, aggregation, concentration, and protein modification (reviewed by Hulse and Cox BioProcess International www(dot)bioprocessintl(dot)com/manufacturing/monoclonal-antibodies/in-vitro-functional-testing-methods-for-monoclonal-antibody-biosimilars-344341).

### SUMMARY OF THE INVENTION

According to an aspect of some embodiments of the present invention there is provided a method of purifying a recombinant antibody having the complementarity determining regions (CDRs) of denosumab, the method comprising subjecting a preparation comprising the antibody to a mixed mode chromatography on a Capto™ Adhere column in a pH range of 6.2-7.4, thereby purifying the antibody.

According to some embodiments of the invention, the pH range is 6.3-7.3.

According to some embodiments of the invention, the pH range is 6.3-7.2.

According to some embodiments of the invention, the pH range is 6.3-7.1.

According to some embodiments of the invention, the pH range is 6.3-7.0.

According to some embodiments of the invention, the pH range is 6.3-6.9.

According to some embodiments of the invention, the pH range is 6.3-6.8.

According to some embodiments of the invention, the pH range is 6.3-6.7.

According to some embodiments of the invention, the pH range is 6.3-6.5.

According to some embodiments of the invention, the pH range is 6.3-6.4.

According to some embodiments of the invention, the pH range is 6.4-7.0.

According to some embodiments of the invention, the pH range is 6.4-6.9.

According to some embodiments of the invention, the pH range is 6.4-6.8.

According to some embodiments of the invention, the pH range is 6.4-6.7.

According to some embodiments of the invention, the pH range is 6.5-6.6.

According to some embodiments of the invention, the subjecting comprises an equilibration buffer and a sample buffer.

According to some embodiments of the invention, the method further comprises subjecting the preparation to an affinity chromatography prior to the subjecting to the mixed mode chromatography.

According to some embodiments of the invention, the method further comprises subjecting the preparation to a cation exchange (CEX) chromatography prior to the subjecting to the mixed mode chromatography.

According to some embodiments of the invention, the subjecting to the CEX chromatography is following the subjecting to the affinity chromatography.

According to some embodiments of the invention, the affinity chromatography comprises a protein A resin.

According to some embodiments of the invention, the protein A resin comprises mAbSelect SuRe™.

According to some embodiments of the invention, the CEX chromatography comprises Eshmuno-S™ resin.

According to some embodiments of the invention, each of the protein A resin and the Eshmuno-S™ resin are packed into a column.

According to some embodiments of the invention, the method further comprises a viral inactivation step prior to the subjecting to the mixed mode chromatography.

According to some embodiments of the invention, the viral inactivation is prior to the CEX chromatography and following the affinity chromatography.

According to some embodiments of the invention, the antibody is expressed in CHO cells.

According to an aspect of some embodiments of the present invention there is provided a composition of matter comprising a purified recombinant antibody having the complementarity determining regions (CDRs) of denosumab obtainable by the method as described herein, wherein the antibody has a binding affinity to FcγRIIa which is about the same as the binding of Xgeva® or Prolia®, as determined by BLI using the parameters of Example 1.

According to an aspect of some embodiments of the present invention there is provided a method of qualifying a recombinant antibody batch comprising an antibody having the complementarity determining regions (CDRs) of denosumab, the method comprising determining a binding affinity of the antibody obtainable according to the method as described herein to an Fc receptor, wherein a binding affinity is within the similarity range for similarity determined from 5 Xgeva® and Prolia® lots and inter assay variability as determined by BLI, is indicative that the antibody batch is a denosumab biosimilar.

According to some embodiments of the invention, the Fc Receptor is FcγRIIa and the affinity is determined using the parameters to Example 2.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart showing the downstream purification for a clarified harvest of denosumab antibody produced according to the present teachings and designated “URI-22”.

FIGS. 2A-F are sensorgrams showing URI-22 binding to FcγRIIa following purification with Capto adhere at different pHs: FIG. 2A—5.5, FIG. 2B—6.1, FIG. 2C—6.6, FIG. 2D—Xgeva®, FIG. 2E—7.5, FIG. 2F—7.8.

### DESCRIPTION OF SPECIFIC EMBODIMENTS OF THE INVENTION

The present invention, in some embodiments thereof, relates to methods of purifying and qualifying antibodies.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

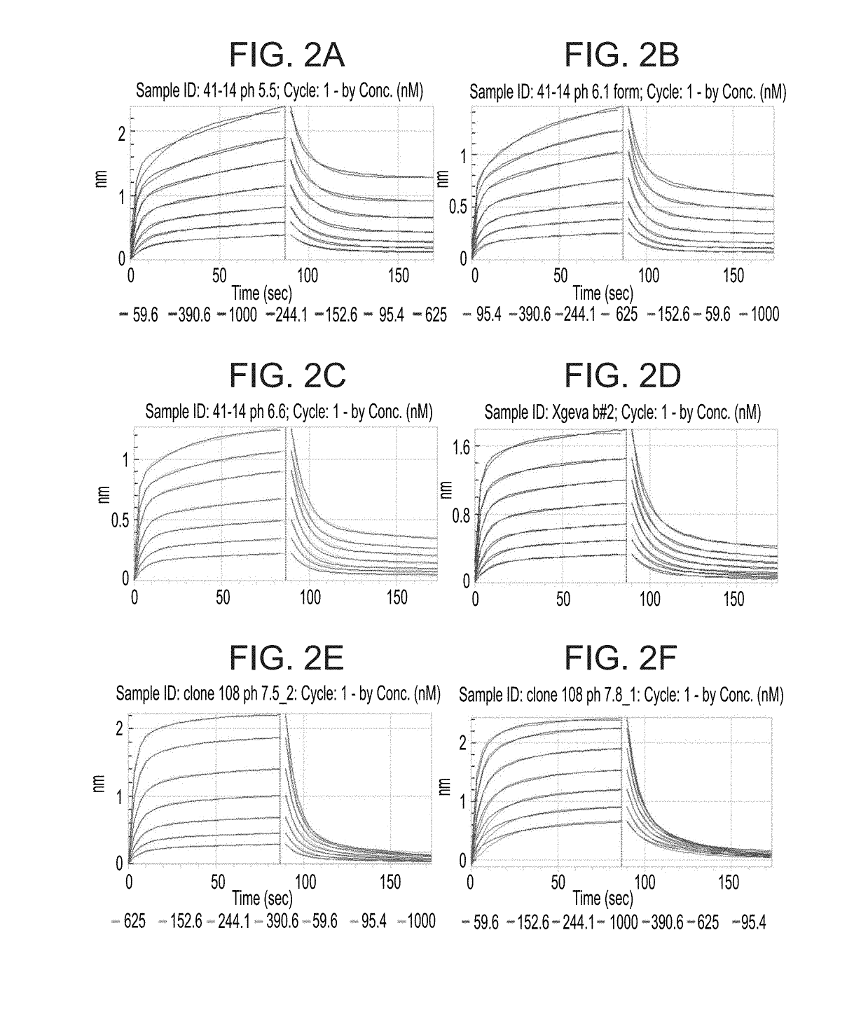

In the process of developing a denosumab biosimilar and more specifically purifying the antibody, the present inventor has realized that the pH at the mixed mode (MM) chromatography stage may affect the antibody conformation, thereby affecting binding of the antibody to target Fc receptors. As shown in FIGS. 2A-F and Table 1 hereinbelow, various binding kinetics are affected by the pH of MM chromatography whereby KD1, KD2 and Kdis2 are the most prominent. Hence an optimal pH range is set for 6.2-7.4 in order to generate a true biosimilar.

Thus, according to an aspect of the invention there is provided a method of purifying a recombinant antibody having the complementarity determining regions (CDRs) of denosumab, the method comprising subjecting a preparation comprising said antibody to a mixed mode chromatography on a Capto™ Adhere column in a pH range of 6.2-7.4, thereby purifying the antibody.

As used herein “denosumab” relates to the generic, compendial, nonproprietary, or official FDA name for the product marketed as Xgeva® or Prolia® by Amgen Inc. and a product that is interchangeable with or equivalent to the product marketed as Xgeva® or Prolia®.

As used herein “a recombinant antibody having the CDRs of denosumab” refers to a denosumab biosimilar (biobetter) having the same CDR composition as that of Xgeva®. According to a specific embodiment, the antibody having the CDRs of denosumab, is an intact antibody having the same frameworks and constant regions as that of Xgeva®. According to an embodiment of the invention, some amino acid alterations may take place however these are typically in the non-CDR region of the antibody.

Thus, embodiments of the invention also contemplates a recombinant antibody having the CDRs of denosumab which have amino acid sequences at least 90%, at least 91%, at least 92%, at least 93%, at least 94%, at least 95%, at least 96%, at least 97%, at least 98%, at least 99%, identical to those of Xgeva® or Prolia®. When the antibodies are not 100% identical to those described herein above, it is conceived that they may comprise either conservative or non-conservative amino acid changes.

According to some embodiments, the antibody has the amino acid sequences of SEQ ID NOs: 1 and 2 (encodable by SEQ ID NOs: 3-4, respectively), also termed URI22.

The term “antibody” refers to an immunoglobulin molecule which comprises four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CHI, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. CDR regions can be determined using methods which are well known in the art, e.g., Kabat numbering scheme.

According to one embodiment, the antibody is not an antibody fragment comprised solely of the antigen binding portion, but also comprises an Fc region. Thus, for example the antibody does not consist solely of (i) a Fab fragment, a monovalent fragment comprising the VL, VH, CL and CHI domains; (ii) a F(ab′)2 fragment, a bivalent fragment comprising two Fab fragments linked by a disulfide bridge at the hinge region; (iii) a Fd fragment comprising the VH and CHI domains; (iv) a Fv fragment comprising the VL and VH domains of a single arm of an antibody, (v) a dAb fragment which comprises a VH domain; or (vi) an isolated complementarity determining region (CDR).

The antibody may be monospecific, (i.e. recognize a single antigen) or bispecific (each arm of the antibody recognizing a different antigen).

The antibody may be of any class e.g. IgAi, IgA₂, IgD, IgE, IgG₁, IgG₂, IgG₃, IgG₄, and IgM antibodies, although preferably the Ab is one which binds to Protein A.

Preferably the antibody is in the class IgG—e.g. IgG2.

As used herein, the term “biosimilar” refers to a biopharmaceutical which is deemed to be comparable in quality, safety, and efficacy to reference product marketed by an innovator company.

The antibody, according to the invention, is typically produced by recombinant means.

According to some aspects the antibody is a humanized or human-type antibody by genetic recombination

To express an antibody recombinantly, a host cell is transfected with one or more recombinant expression vector carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N. Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the term “transfection” are intended to encompass a wide variety of techniques commonly used for the introduction of exogenous DNA into aprokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescens, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24, 178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fraitfly), and Bombyx mori have been identified. A variety of viral strains for transfection are publicly available, e.g., the L-I variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N. Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In certain embodiments it may be desirable to transfect a host cell with DNA encoding either the light chain or the heavy chain (but not both) of an antibody of this invention (i.e., having the CDRs of denosumab). Thus, for example, the light chain and heavy chain may be expressed in inclusion bodies in bacterial cultures and subsequently refolded.

When using recombinant techniques, the antibody can be produced intracellularly, in the periplasmic space, or directly secreted into the medium. If the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon™ or Millipore Pellicon™ ultrafiltration unit.

Lysis of the cells may be performed by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration.

The antibodies described herein are purified from a preparation comprising impurities.

According to an embodiment, the purified preparation comprises aggregates %<0.9%, residual protein A (ppm)<1.

As used herein, a “preparation” comprises the recombinant antibody having the CDRs of denosumab and one or more contaminant, i.e., impurities. The preparation can be obtained directly from a host cell (e.g., CHO cells) or organism producing the polypeptide. Without intending to be limiting, examples of preparations that can be purified according to a method of the present invention include harvested cell culture fluid, cell culture supernatant and conditioned cell culture supernatant.

According to one embodiment, the impurity is a protein aggregate.

As used herein, the term “protein aggregate” refers to multimers (such as dimers, tetramers or higher order aggregates) of the Ab to be purified and may result e.g. in high molecular weight aggregates.

According to a specific embodiment, the preparation comprises a Chinese hamster ovary cell protein, referred to herein as “CHOP”. The amount of CHOP present in a preparation comprising the antibody having the CDRs of denosumab provides a measure of the degree of purity for the protein of interest. Typically, the amount of CHOP in a protein preparation is expressed in parts per million relative to the amount of the protein of interest in the preparation. It is understood that where the host cell is another cell type, e.g., a eukaryotic cell other than CHO cells, an insect cell, or a plant cell, of a yeast cell, host cell protein (HCP) refers to the proteins, other than target protein, found in a lysate of the host cell.

Preparations can, for example, be aqueous solutions, organic solvent systems, or aqueous/organic solvent preparations or solutions. The preparations are often complex preparations or solutions comprising many biological molecules (such as proteins, antibodies, hormones, and viruses), small molecules (such as salts, sugars, lipids, etc.) and even particulate matter. While a typical preparation of biological origin may begin as an aqueous solution or suspension, it may also contain organic solvents used in earlier separation steps such as solvent precipitations, extractions, and the like. Examples of preparations that may contain valuable biological substances amenable to the purification by various embodiments the present invention include, but are not limited to a harvested cell culture fluid, a cell culture supernatant, a conditioned cell culture supernatant from a bioreactor, a homogenized cell suspension, plasma, plasma fractions and milk.

According to one embodiment, the preparation has already been subjected to a chromatography step, e.g., affinity chromatography, non-affinity chromatography (e.g., cation exchange chromatography) as described hereinbelow and in the Examples section which follows.

According to one embodiment, the preparation has been first clarified (as shown in Example 1 for small scale purification).

As used herein, the term “clarified” refers to a sample (i.e. a cell suspension) having undergone a solid-liquid separation step involving one or more of centrifugation, microfiltration and depth filtration to remove host cells and/or cellular debris. A clarified fermentation broth may be a cell culture supernatant. Clarification is sometimes referred to as a primary or initial recovery step and typically occurs prior to any chromatography or a similar step. In other cases some chromatography has been applied to the preparation.

Following generation of the recombinant antibody, purification steps are taken so as to decrease the level of impurities in the preparation.

According to an embodiment of the invention, mixed mode chromatography is taken after the preparation has been subjected to affinity chromatography (see Example 1).

According to an additional or an alternative embodiment, cation exchange chromatography is taken prior to MM chromatography and optionally following affinity chromatography.

It will be appreciated that intervening steps may be taken during the purification procedure and also following the purification procedure.

Examples of contemplated intervening steps include viral inactivation and filtration, as further described herein below.

The term “chromatography resin” or “chromatography media” are used interchangeably herein and refer to any kind of solid phase which separates an analyte of interest (e.g., an Fc region containing protein such as an immunoglobulin) from other molecules present in a preparation. Usually, the analyte of interest is separated from other molecules as a result of differences in rates at which the individual molecules of the preparation migrate through a stationary solid phase under the influence of a moving phase, or in bind and elute processes. Non-limiting examples include cation exchange resins, affinity resins and mixed mode resins. The volume of the resin, the length and diameter of the column to be used, as well as the dynamic capacity and flow-rate depend on several parameters such as the volume of fluid to be treated, concentration of protein in the fluid to be subjected to the process of the invention, etc. Determination of these parameters for each step is well within the average skills of the person skilled in the art.

Affinity Resin:

In certain embodiments, the preparation is subjected to affinity chromatography to purify the antibody (i.e., having the CDRs of denosumab) away from impurities. In certain embodiments the chromatographic material is capable of selectively or specifically binding to the antibody of interest. Non-limiting examples of such chromatographic material include: Protein A, Protein A/G, Protein G and Protein L chromatographic material. In specific embodiments, the affinity chromatography step involves subjecting the primary recovery sample to a column comprising a suitable Protein A resin. Protein A resin is useful for affinity purification and isolation of a variety of antibody isotypes, particularly I_{gG1}, IgG₂, and IgG₄. Protein A is a bacterial cell wall protein that binds to mammalian IgGs primarily through their Fc regions. In its native state, Protein A has five IgG binding domains as well as other domains of unknown function. There are several commercial sources for Protein A resin including, but not limited to, MabSelect SuRe™, MabSelect SuRe LX, MabSelect, MabSeiect Xtra, rProtein A Sepharose from GE Healthcare, ProSep HC, ProSep Ultra, and ProSep Ultra Plus from EMD Millipore, MapCapture from Life Technologies. According to a specific embodiment, the protein A resin is MabSelect SuRe™.

In certain embodiments, the Protein A column can be equilibrated with a suitable buffer prior to sample loading. Such a buffer typically has the same properties as the clarified harvest e.g. pH±0.2, conductivity ±2 mS/cm. A non-limiting example of a suitable buffer is a 20 mM phosphate buffer, 150 mM NaCl, pH 7.4. Following this equilibration, the sample can be loaded onto the column. Preferably, the load should have a minimum residence time NLT of about 2-30 minutes. According to some embodiments a minimum residence time NLT should be between 1-10 minutes or 1-5 minutes. In certain embodiments, the preparation is loaded onto the affinity resin at a loading concentration of 10-80, 20-60, 30-50 or more preferably between 20-40 mg of antibody per ml of resin. According to a specific embodiment a loading buffer of 20 mM phosphate buffer, 150 mM NaCl, pH 7.4 is used.

Following the loading of the column, the column can be washed one or multiple times using, e.g., the equilibrating buffer (e.g. a buffer comprising 20 mM sodium phosphate buffer, 150 mM NaCl). Other washes, including washes employing different buffers, can be employed prior to eluting the column. For example, the column can be washed using one or more column volumes of 20 mM sodium phosphate buffer, 1 M NaCl, and/or 100 mM acetate buffer pH 5.0. This wash can optionally be followed by one or more washes using the equilibrating buffer.

The Protein A column can then be eluted using an appropriate elution buffer. A non-limiting example of a suitable elution buffer is an acetic acid buffer, pH of about 3.2-3.6. Suitable conditions are, e.g., 0.1 M acetic acid, pH of about 3.2.

The eluate can be monitored using techniques well known to those skilled in the art. For example, the absorbance at OD₂₈₀ can be followed. Column eluate can be collected starting with an initial deflection of about 40 mAU to a reading of about 40 mAU at the tailing edge of the elution peak. The elution fraction(s) of interest can then be prepared for further processing.

An exemplary height of an affinity column (e.g. Mab SelectSure) is between 10-24 cm. An exemplary resident time on an affinity column (e.g. Mab SelectSure) is less than 15 minutes, for example about 3 minutes.

An exemplary affinity chromatography protocol is provided in Example 2 of the Examples section which follows and is incorporated into this section by reference in its entirety.

Cation Exchange Resin:

Following affinity chromatography, the antibody may be further purified using a cation exchange resin (CEX). In performing the separation, the antibody sample preparation can be contacted with the cation exchange material by using any of a variety of techniques, e.g., using a batch purification technique or a chromatographic technique. For the purification of an antibody, the antibody must have a charge opposite to that of the functional group attached to the ion exchange material, e.g., resin, in order to bind. For example, antibodies, which have an overall positive charge when present in a buffer having a pH below the antibody's pI, will bind well to cation exchange material, which contain negatively charged functional groups. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). Cationic substituents may be attached to matrices in order to form cationic supports for chromatography. Non-limiting examples of cationic exchange substituents include carboxymethyl (CM), sulfoethyl(SE), sulfopropyl(SP), phosphate(P) and sulfonate(S).

Preferably, the CEX resin comprises a SO₃ functional group.

Cellulose ion exchange resins such as DE23™, DE32™, DE52™, CM-23™, CM-32™, and CM-52™ are available from Whatman Ltd. Maidstone, Kent, U.K. SEPHADEX®-based and cross-linked ion exchangers are also known. For example, DEAE-, QAE-, CM-, and SP-SEPHADEX® and DEAE-, Q-, CM- and S-SEPHAROSE® and SEPHAROSE® Fast Flow are all available from Pharmacia AB. Further, both DEAE and CM derivatized ethylene glycol-methacrylate copolymer such as TOYOPEARL™ DEAE-650S or M and TOYOPEARL™ CM-650S or M are available from Tosoh, Philadelphia, Pa.

Further, both DEAE and CM derivatized ethylene glycol-methacrylate copolymer such as TOYOPEARL™ DEAE-650S or M and TOYOPEARL™ CM-650S or M are available from Tosoh, Philadelphia, Pa., or Nuvia S and U OSphere™ S from BioRad, Hercules, Calif., Eshmuno® S from EMD Millipore, Billerica, Calif.

According to a particular embodiment, the CEX resin comprises Eshmuno® S.

According to another embodiment, the CEX resin comprises Fractogel EMD COO⁻ (M).

According to a particular embodiment, the column is washed prior to equilibration with a high salt buffer—e.g. 20 mM acetate buffer pH of about 5.

According to a specific embodiment, the loading buffer is comprised from protein A eluate titrated to pH 3.6 by 0.5M acetic acid, this intermediate is titrated to pH 5.0, before loading the intermediated is filtered. Protein concentration may vary from 3-10 mg/mL.

The CEX resin may be washed prior to eluting e.g. using the equilibration buffer at pH 5.

An additional step of washing can be done at higher pH (e.g., 6) with e.g., 10 CV “column volume”.

The antibody may then elute with a gradient of 0 to 100% of 0.3 M sodium chloride in 20 mM phosphate buffer pH 6.0.

The cation exchange procedure can be carried out at or around room temperature.

An exemplary CEX chromatography protocol is provided in Example 2 of the Examples section which follows and is incorporated into this section by reference in its entirety.

Mixed Mode Chromatography:

As mentioned, the antibody is subjected to mixed mode (MM) purification. Mixed mode chromatography is chromatography that utilizes a mixed mode media, such as, but not limited to Capto Adhere™ available from GE Healthcare. Such a media comprises a mixed mode chromatography ligand. In certain embodiments, such a ligand refers to a ligand that is capable of providing at least two different, but co-operative, sites which interact with the substance to be bound. One of these sites gives an attractive type of charge-charge interaction between the ligand and the substance of interest. The other site typically gives electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole etc.

In certain embodiments, the mixed-mode resin comprises a negatively charged part and a hydrophobic part. In one embodiment, the negatively charged part is an anionic carboxylate group or anionic sulfo group for cation exchange. Examples of such supports include, but are not limited to, Capto Adhere® (GE Healthcare). Capto Adhere® is a strong anion exchanger with multimodal functionality which confers different selectivity to the resin compared to traditional anion exchangers. The Capto Adhere® ligand (N-Benzyl-N-methyl ethanolamine) exhibits multiple modes of protein-interactive chemistries, including ionic interaction, hydrogen bonding and hydrophobic interaction. The multimodal functionality of the resin confers it with an ability to remove antibody dimers and aggregates, leached protein A, host cell proteins (HCP), antibody/HCP complexes, process residuals and viruses. The resin may be used in flowthrough mode in the context of a production scale polishing step employing operational parameters designed to have the mAb pass directly through the column while the contaminants are adsorbed.

The present inventor has realized that the pH at the MM chromatography stage may possibly affect the antibody conformation, affecting binding of the antibody to target Fc receptors. As shown in FIGS. 2A-F and Table 1 hereinbelow, various binding kinetics are affected by the pH of MM chromatography whereby D1, D2 and Kdis2 are the most prominent. Hence an optimal pH range is set for 6.2-7.4 in order to generate the biosimilar.

For the purification of the antibody, the MM resin may be equilibrated prior to loading with an equilibration buffer which is substantially (about) the same in pH and conductivity or the same as the loading buffer in which the antibody is loaded.

Thus, according to a specific embodiment, when referring to subjecting to MM chromatography, both the equilibration buffer, the loading buffer and the washing buffer are of the contemplated pH range. The buffers may have identical or non-identical pHs as long as they are within the contemplated range as per below.

According to a specific embodiment, the pH range is 6.3-7.3.

According to a specific embodiment, the pH range is 6.3-7.2.

According to a specific embodiment, the pH range is 6.3-7.1.

According to a specific embodiment, the pH range is 6.3-7.0.

According to a specific embodiment, the pH range is 6.3-6.9.

According to a specific embodiment, the pH range is 6.3-6.8.

According to a specific embodiment, the pH range is 6.3-6.7.

According to a specific embodiment, the pH range is 6.3-6.5.

According to a specific embodiment, the pH range is 6.3-6.4.

According to a specific embodiment, the pH range is 6.4-7.0.

According to a specific embodiment, the pH range is 6.4-6.9.

According to a specific embodiment, the pH range is 6.4-6.8.

According to a specific embodiment, the pH range is 6.4-6.7.

According to a specific embodiment, the pH range is 6.5-6.6.

According to a particular embodiment, the column is equilibrated with 20 mM phosphate buffer, pH 6.5-6.6.

According to an exemplary embodiment, the Ab may be loaded onto the MM resin in a loading buffer comprising 20-50 mM phosphate buffer e.g., 100 mg of protein per ml of the resin Typically, the loading buffer, the equilibration buffer and the washing buffer are adjusted to a pH of about 6.5-6.6, prior to loading. According to a specific embodiment, the loading density is 3-7 mg/ml and the conductivity if 5-10 mD/cm. Various buffers can be employed including citrate phosphate, TRIS HCl, HEPES etc.

The MM exchange procedure can be carried out at or around room temperature.

An exemplary height of a mixed mode column (e.g. Capto Adhere) is between 10-20 cm.

An exemplary MM chromatography protocol is provided in Example 2 of the Examples section which follows and is incorporated into this section by reference in its entirety.

As mentioned, the antibody may be subjected to a viral inactivation step. This may be performed at any stage during the purification procedure. According to one embodiment, the viral inactivation is effected between the affinity purification step and the CEX purification step or alternatively following the MM step.

An exemplary viral inactivation protocol is provided in Example 2 of the Examples section which follows and is incorporated into this section by reference in its entirety.

The phrase “viral inactivation”, as used herein, refers to a decrease in the activity of adventitious enveloped viruses in a particular sample (“inactivation”). Such decreases in the activity of enveloped viruses can be on the order of about 3 log reduction factor (LRF) preferably of about 4 LRF, more preferably of about 5 LRF, even more preferably of about 6 LRF.

Any one or more of a variety of methods of viral inactivation can be used including heat inactivation (pasteurization), pH inactivation, solvent/detergent treatment, UV and γ-ray irradiation and the addition of certain chemical inactivating agents such as β-propiolactone or e.g., copper phenanthroline as in U.S. Pat. No. 4,534,972, the entire teaching of which is incorporated herein by reference.

Methods of pH viral inactivation include, but are not limited to, incubating the preparation for a period of time at low pH, and subsequently neutralizing the pH. In certain embodiments the preparation will be incubated at a pH of between about 2 and 5, preferably at a pH of between about 3 and 4, and more preferably at a pH of about 3.6. The pH of the sample preparation may be lowered by any suitable acid including, but not limited to, citric acid, acetic acid, caprylic acid, or other suitable acids. The choice of pH level largely depends on the stability profile of the antibody product and buffer components. It is known that the quality of the target antibody during low pH virus inactivation is affected by pH and the duration of the low pH incubation. In certain embodiments the duration of the low pH incubation will be from 0.5 hr to 2 hr, preferably 0.5 hr to 1.5 hr, and more preferably the duration will be about 1 hr. Virus inactivation is dependent on these same parameters in addition to protein concentration, which may limit inactivation at high concentrations. Thus, the proper parameters of protein concentration, pH, and duration of inactivation can be selected to achieve the desired level of viral inactivation.

In certain embodiments viral filtration is performed. This can be achieved via the use of suitable filters. A non-limiting example of a suitable filter is the Ultipor DV50™ filter from Pall Corporation. In certain embodiments, alternative filters are employed for viral inactivation, such as, but not limited to, Sartorius filters, Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, 111.).

In those embodiments where viral inactivation is employed, the sample preparation can be adjusted, as needed, for further purification steps. For example, following low pH viral inactivation the pH of the sample preparation is typically adjusted to a more neutral pH, e.g., from about 4 to about 8, and preferably about 5, prior to continuing the purification process. Additionally, the preparation may be flushed with water for injection (WFI) to obtain a desired conductivity.

An exemplary viral inactivation protocol is provided in Example 2 of the Examples section which follows and is incorporated into this section by reference in its entirety.

Certain embodiments of the present invention employ filtration prior to loading of a sample onto a column. Thus, for example, the present invention contemplates passing the sample over a filter (for example a 0.2 μm filter) prior to loading on to a cation exchange column or a mixed mode column. Examples of such filters include hydrophilic DURAPORE PVDF or PES polyethersulfone filters.

Certain embodiments of the present invention employ ultrafiltration and/or diafiltration steps to further purify and concentrate the antibody sample. Typically, this is carried out following the MM purification step. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size that allow transfer of protein with average size of 50 kDa (for example) or smaller. By employing filters having such small pore size, the volume of the sample can be reduced through permeation of the sample buffer through the filter while antibodies are retained behind the filter.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultrafiltered at a rate approximately equal to the ultratfiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody.

The novel methods of purifying the antibodies described in the present application result in the antibodies themselves also being novel.

Thus, there is provided a composition of matter comprising a purified recombinant antibody having the complementarity determining regions (CDRs) of denosumab obtainable by the method as described herein, wherein said antibody has a binding affinity to FcγRIIa which is about the same as the binding of Xgeva® or Prolia®, as determined by BLI using the parameters of Example 1.

Also provided is a method of qualifying a recombinant antibody batch comprising an antibody having the complementarity determining regions (CDRs) of denosumab, the method comprising determining a binding affinity of the antibody obtainable as described herein to an Fc receptor, wherein a binding affinity similar to that of Xgeva® or Prolia®, as determined by BLI, is indicative that the antibody batch is a denosumab biosimilar.

As used herein “similar” or “about the same” refers to a binding affinity that is within the target range for similarity determined from inter lot variability of at least 5 Xgeva® and Prolia® lots and from inter-assay variability.

As used herein “Fc receptor” or FcR refers to a protein found on the surface of certain cells—including, among others, B lymphocytes, follicular dendritic cells, natural killer cells, macrophages, neutrophils, eosinophils, basophils, human platelets, and mast cells—that contribute to the protective functions of the immune system.

According to a specific embodiment the FcR is an Fey receptor e.g., FcγRIIa/FcγRIIA. However, the binding to other Fc receptors is also contemplated in the process of qualification as described herein.

All of the Fey receptors (FcγR) belong to the immunoglobulin superfamily and are the most important Fc receptors for inducing phagocytosis of opsonized microbes. This family includes several members, FcγRI (CD64), FcγRIIA (CD32), FcγRIIB (CD32), FcγRIIIA (CD16a), FcγRIIIB (CD16b), which differ in their antibody affinities due to their different molecular structure. For instance, FcγRI binds to IgG more strongly than FcγRII or FcγRIII does. FcγRI also has an extracellular portion composed of three immunoglobulin (Ig)-like domains, one more domain than FcγRII or FcγRIII has. This property allows FcγRI to bind a sole IgG molecule (or monomer), but all Fcγ receptors must bind multiple IgG molecules within an immune complex to be activated.

The Fc-gamma receptors differ in their affinity for IgG and likewise the different IgG subclasses have unique affinities for each of the Fc gamma receptors. These interactions are further tuned by the glycan (oligosaccharide) at position CH2-84.4 of IgG. For example, by creating steric hindrance, fucose containing CH2-84.4 glycans reduce IgG affinity for FcγRIIIA In contrast, GO glycans, which lack galactose and terminate instead with GlcNAc moieties, have increased affinity for FcγRIIIA

Another FcR is expressed on multiple cell types and is similar in structure to MHC class I. This receptor also binds IgG and is involved in preservation of this antibody. However, since this Fc receptor is also involved in transferring IgG from a mother either via the placenta to her fetus or in milk to her suckling infant, it is called the neonatal Fc receptor (FcRn). Recently, research suggested that this receptor plays a role in the homeostasis of IgG serum levels.

Fc-alpha receptors—Only one Fc receptor belongs to the FcαR subgroup, which is called FcαRI (or CD89). FcαRI is found on the surface of neutrophils, eosinophils, monocytes, some macrophages (including Kupffer cells), and some dendritic cells. It is composed of two extracellular Ig-like domains, and is a member of both the immunoglobulin superfamily and the multi-chain immune recognition receptor (MIRR) family. It signals by associating with two FcRγ signaling chains.^{[10]} Another receptor can also bind IgA, although it has higher affinity for another antibody called IgM. This receptor is called the Fc-alpha/mu receptor (Fcα/μR) and is a type I transmembrane protein. With one Ig-like domain in its extracellular portion, this Fc receptor is also a member of the immunoglobulin superfamily.

Two types of FcεR are known: the high-affinity receptor FcεRI is a member of the immunoglobulin superfamily (it has two Ig-like domains). FcεRI is found on epidermal Langerhans cells, eosinophils, mast cells and basophils. As a result of its cellular distribution, this receptor plays a major role in controlling allergic responses. FcεRI is also expressed on antigen-presenting cells, and controls the production of important immune mediators called cytokines that promote inflammation; the low-affinity receptor FcεRII (CD23) is a C-type lectin. FcεRII has multiple functions as a membrane-bound or soluble receptor; it controls B cell growth and differentiation and blocks IgE-binding of eosinophils, monocytes, and basophils.

To measure Fc binding affinity BLI technology is used. Independently prepared dilutions of test samples and control-originator's denosumab, are tested at a number of concentrations (e.g., 6) for binding to affinity (e.g., His-tag) labeled FcR e.g., FcγRIIa immobilized on e.g., Ni-NTA biosensors. Binding affinity parameters are calculated using a 2:1 heterogeneous ligand model fit by the Octet software. It will be appreciated that other binding methods can also be used, e.g., SPR, ELISA.

One of ordinary skill in the art would know which BLI parameters should be employed and which binding kinetics are to be determined.

The following abbreviations are used herein and each of them can be used for determining the binding parameter as described herein. KD1 affinity constant 1, KD2-affinity constant2 KDIS-dissociation constant, Kon-association constant, SSKD-steady-state KD, relative response-response, obtained for binding to FcγRIIa at each concentration of the sample relative to the control, calculated using the PLA software with a linear line analysis.

According to a specific embodiment, the antibody generated according to the According to a specific embodiment, the antibody generated according to the present teachings exhibits binding kinetics to the test FcR which are about the same as Xgeva® or Prolia. Within the target range for comparability determined from 5 Xgeva® and Prolia® lots and inter-assay variability.

Typically, binding to the Fc receptor is determined following the MM stage. However it will be appreciated that binding to Fc receptors may also determined at other stages; from clarified harvests to partially purified samples.

According to a specific embodiment, the Fc Receptor is FcγRIIa and said BLI is determined using the parameters to Example 2. Exemplary values are provided in Example 2.

Antibodies obtained using the process of the invention may be incorporated into pharmaceutical compositions suitable for administration to a subject. Typically, the pharmaceutical composition comprises an antibody and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of antibody, or antigen-binding portion thereof.

Pharmaceutical compositions comprising antibodies purified using the methods of the invention may be found in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by subcutaneous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, or antigen-binding portion thereof) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Supplementary active compounds can also be incorporated into the compositions. In certain embodiments, an antibody, or antigen-binding portion thereof, for use in the methods of the invention is co-formulated with and/or co-administered with one or more additional therapeutic agents

There are various indications for which denosumab is contemplated, some are already approved and some are still under research, all these and also future indications are contemplated here for the compositions described herein both for adult and pediatric use.

Examples include but are not limited to giant cell tumor, osteoporosis, aromatase inhibitor-induced bone loss, androgen deprivation induced bone loss, skeletal related events (e.g., bone fractures, pain), hypercalcemia of malignancy and any other indication for which RANKL serves as a target.

It is expected that during the life of a patent maturing from this application many relevant indication for which RANKL serves as a target will be developed and the scope of the term indication for which RANKL serves as a target is intended to include all such new technologies a priori

As used herein the term “about” refers to ±20% or 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including preparations thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 3 is expressed in a DNA sequence format (e.g., reciting T for thymine), but it can refer to either a DNA sequence that corresponds to a DNA nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Cloning of URI22.

URI22 (Insight-denosumab) expression vector pDU22KTDG1 is a pDRIVE based triple plasmid containing the kappa and gamma expression cassettes controlled by the CAG promoter and a dhfr expression cassette controlled by the Tk promoter. Uri22 Kappa coding sequence was constructed by combining a 390 bp optimized synthetic fragment of the variable region and an Fc fragment amplified from spleen cDNA. Uri22 Gamma fragment was constructed from two fragments, an optimized synthetic fragment encoding the variable sequence and a signal peptide and an IgG2 Fc fragment amplified from spleen cDNA. The TKDHFR fragment combined from 2 fragments: a TK promoter and partial DHFR fragment restricted from an Insight custom design pRTD2 and a PCR, restriction site modified, DHFR. The three expression cassettes were inserted into the pDRIVE-CAG in tandem in a clockwise orientation, Uri22 Kappa, TKDHFR and then Uri22 Gamma. The sequences of the kappa and the gamma cDNAs composing denosumab and the corresponding gamma and the kappa amino acid sequences are presented in SEQ ID NOs: 1-3 (URI22 Kappa and Gamma).

Clone Establishment

Duk⁻ cells (CHO/dhfr⁻, ATCC # CRL-9096) were transfected with InSight-denosumab expression vector pDU22KTDG1 by electroporation. Following transfection, cells pools were selected in ProCHO5 medium without HT, followed by four steps amplification: 200 nM MTX, 500 nM, 1 μM MTX followed by 5 μM MTX.

Following recovery pools are seeded in a semi-solid medium for subsequent picking of highly expressing cells by the Clonepix system.

### Example 1

### Small—Scale Purification of Denosumab (Insight, URI-22) Clarified Harvests

In the protocol below, an example of purification of InSight-denosumab clarified harvest samples using Capto adhere chromatography at pH 6 is presented.

For purification at different Capto adhere pH, the 28 μl volume of 0.5 M phosphate dibasic, added to the MabSelect sure eluate (eluate of MabSelect Sure, listed below) collection plate, is adjusted to allow equilibration of the eluate to the desired pH (pH 6). In addition, the pH of the Capto adhere equilibration buffer is adjusted accordingly to this pH.

Materials:

A. Mab Select Purification:

1. PreDictor Mab select Sure 96 wells plate, 50 μl (GE Healthcare Cat#28-9258-25).

2. Wash buffer-20 mM phosphate buffer, 150 mM NaCl, pH 7.4.

3. Elution buffer-25 mM Citrate buffer pH 3.1.

4. 1 M NaCl.

5. 0.5 M phosphate dibasic.

6. 96-well, 0.5 ml, U-bottom polypropylene plate (Agilent Technologies Cat#5042-1386).

B. Capto Adhere Purification:

1. PreDictor Capto adhere 96 wells plate, 50 μl (GE Healthcare Cat#28-9258-19).

2. Equlibration buffer-18 mM citrate, 53 mM phosphate, 166 mM Nacl, buffer pH 6.

3. 96-well, 0.5 ml, U-bottom polypropylene plate (Agilent Technologies Cat#5042-1386).

Method:

All filtration steps are performed by centrifugation at 500-600×g.

A. MabSelect sure purification

1. Suspend MabSelect sure media particles by shaking, as recommended in GE Instructions#28-9258-34, and then remove storage buffer.

2. After storage buffer removal from MabSelect plate, wash each well 3 times with 200 μl wash buffer (20 mM phosphate buffer, 150 mM NaCl, pH 7.4).

3. Apply clarified harvests at a volume equivalent to 0.8 mg IgG (two wells per sample) and incubate with head-over-tail rotation for 20 min.

4. Wash 3 times with 200 μl of wash buffer.

5. Prepare a collection plate for collecting sample eluates. For each sample, pipette 46 μl 1M NaCl and 28 μl 0.5M phosphate Dibasic (to naturalize pH of the eluate from 3.1 to 6) to all wells corresponding to the sample wells in the collection plate.

B. Elute sample by adding 200 μl 25 mM Citrate buffer to the MabSelect sure plate and filtration by centrifugation at 500-600×g.

C. Capto adhere purification

4. Equilibrate Capto adhere plate with 200 μl equilibration buffer.

5. 18 mM citrate, 53 mM phosphate, 166 mM Nacl, buffer pH 6.

6. and incubate with head-over-tail rotation for 20 min.

7. Remove equilibration buffer by centrifugation at 58-84×g.

8. Apply MabSelect sure to the Capto adhere plate wells and incubate with head-over-tail rotation for 20 min. Collect flowthrough from Capto adhere resin by filtration into a new collection plate.

### Example 2

### Full Scale Purification of Denosumab (Insight, URI-22) Clarified Harvests

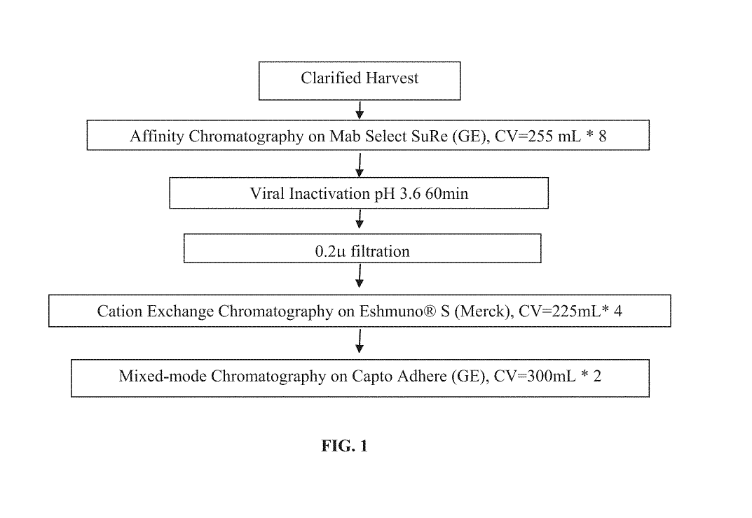

The protocol is schematically illustrated in FIG. 1.

The below provides the experimental procedures of lab scale purification.

### Purification of InSight-Denosumab (URI-22) by Affinity Chromatography on a MabSelect SuRe Column

Column set-up

Attach MabSelect Sure column (GE Healthcare) to a chromatographic system.

Set the maximal pressure at 0.3 MPa.

Wash the column (removing the 20% Ethanol) in “down flow” direction with >3 CV of water at flow rate of 500 cm/h.

Column run

Wash the column with 5 CV of 20 mM phosphate buffer, 150 mM NaCl, pH 7.4 at a flow rate of 500 cm/h in down flow mode.

Load the clarified harvest containing 1.4-3.4 mg/mL, a maximum InSight-denosumab (32 mg/mL resin).

Note:
Minimum residence time should be NLT 3 minutes.

Wash the column with 20 mM phosphate buffer, 150 mM NaCl, pH 7.4, at the same flow rate preformed during the loading stage.

When the absorbance at 280 nm returns to a low and stable reading, continue to wash the column with 20 mM Phosphate buffer, 150 mM NaCl, pH 7.4, at a linear flow rate of 500 cm/hour up to 10 CV from the beginning of the wash step (previous step).

Elute InSight denosumab with 100 mM acetate buffer pH 3.2, at a linear flow rate of ˜250 cm/hour.

Collect the InSight denosumab peak Start collecting when the absorbance at 280 nm reaches 40 mAU OD 280 nm.

Stop collecting when absorbance value returns to 40 mAU OD 280 nm.

Viral Inactivation Step

Note:
The procedure is performed at 18-22° C.

Lowering the pH level.

Place the combined preparation on a magnetic stirrer.

Start stirring gently the solution and measure pH level and conductivity.

Start lowering pH level slowly to pH 3.6 (minimal) with 0.5 M acetic acid (start pH pH 4.0-4.2).

Add continuously small amounts of 0.5 M Acetic acid solution while gently stirring.

Continue stirring for a few more minutes and check if pH level is stable.

Measure pH and conductivity.

Store the bottle at room temperature (18-22° C.) for 60 minutes.

Neutralizing the pH to pH 5.0.

Raise the pH level slowly to pH 5.0 with 0.5 M sodium phosphate dibasic.

Alternatively 0.1M NaOH.

### Purification of InSight Denosumab by Cation Exchanger Chromatography on Eshmuno® S Column

Column set-up

Attach column packed with Eshmuno® S resin (Merck)

Wash the column with 3 CV of water at a flow rate of 500 cm/h.

Wash the column with 3 CV of 20 mM phosphate buffer, 1 M Sodium Chloride pH 6.0 at a flow rate of 500 cm/h, until the conductivity and pH are the same as those of the buffer.

Column run

Equilibrate the column with 3 CV of 20 mM acetate buffer pH 5.0 at a flow 500 cm/hour.

Check that the absorbance reading (at 254 nm), pH and the system pressure are stabilized at a flow rate of 150 cm/hour for one CV.

Load InSight denosumab solution 16.5 g at a flow rate of 49 150 cm/hour.

Wash the column with 3 CV of 20 mM acetate buffer pH 5.0 at a flow rate of 150 cm/hour.

Wash the column with 10 CV of 20 mM phosphate buffer, pH 6.0, at a flow rate of 500 cm/h.

Perform auto zero to the absorbance.

Elute InSight denosumab with the following gradient program (Table 1):

TABLE 1

| CV | Flow rate cm/h | (%) “A” | (%) “B” |
|---|---|---|---|
| Initial | 150 | 100 | 00 |
| 20 | 150 | 0 | 100 |

A—20 mM phosphate buffer, pH 6.0.
B—20 mM phosphate buffer, pH 6.0, in 0.3M NaCl.

Start collecting the denosumab peak when the absorbance at 254 nm reaches 400 mAU.

Stop collecting the peak when, on the down slope, the absorbance at 254 nm reaches 400 mAu.

### Purification of InSight Denosumab by Mixed-Mode Chromatography on a Capto Adhere Column

InSight denosumab load should be ˜100 mg/mL Capto Adhere (GE Healthcare) resin.

Titrate the solution, avoiding the formation of foam with 0.1M NaOH or 0.5M phosphate dibasic to pH 6.5.

Dilute the sample with water or 20 mM phosphate buffer pH 6.5.

Column set-up

Attach the column packed with Capto Adhere (GE Healthcare) resin.

Wash the column with 3 CV of water at a flow rate of 230 cm/h.

Wash the column with ˜5 CV of 20-50 mM phosphate buffer, 1 M sodium chloride, pH 6.5 at a flow rate of 230 cm/h, until the conductivity and pH are the same as those of the buffer.

Column run

Equilibrate the column with 5 CV of 20-50 mM Phosphate buffer, pH 6.5, 5 mS/cm±1 at a flow rate of 230 cm/h.

Check that the absorbance reading, pH and the system pressure are stabilized.

Load “Capto Adhere load on the Capto Adhere column (100 mg InSight denosumab/mL resin) at a flow rate of 75 mL/minute (230 cm/h).

Start collecting the unbound fraction containing InSight denosumab when absorbance at 280 nm reaches ˜10 mAu, into a 20 L bottle labeled “Capto-Adhere unbound #4.1”. After loading has ended wash the column with 20-50 mM Phosphate buffer, pH 6.2, 5 mS/cm±1 at a flow rate of 230 cm/h.

Stop collecting the peak when, on the down slope, peak absorbance reaches ˜50 mAU.

Keep the solution cold at 2-8° C.

### Example 3

### Effect of pH of Capto Adhere Chromatography on Binding Affinity Parameters

Using the purification process of Example 1, URI-22 clones were purified by Capto adhere chromatography at various pHs; 5.5, 6.1 and 6.6. In addition, samples of URI-22 clones that underwent full-scale purification with Capto adhere chromatography at pH 6.5, 7.5 and 7.8 were obtained from DSP unit, as in Example 2. Binding of these samples to FcγRIIa was tested using the Octet QK384 with Ni-NTA biosensors.

### Experimental Procedures

FcγRIIa binding assay is based on label free biolayer interferometry (BLI) analysis using the Octet QK384 system at 25° C. and 1000 rpm. His tag-labeled FcγRIIa (R&D Cat#1330-CD-050/CF) (1.5 μg/ml) is bound to nickel coated biosensors. Binding kinetics of denosumab, at six concentrations (47-500 nM), to bound FcγRIIa is measured. Resulting signals are converted to affinity parameters using a 2:1 heterogeneous ligand binding model fit by the Octet analysis software.

Results

FIGS. 2A-F demonstrate the effect of Capto buffer pH during purification on URI-22 clone sensorgram for binding to FcγRIIa as compared to the originator drug-Xgeva®. Table 2 presents binding affinity parameters of these samples to FcγRIIa. Table 2 and FIGS. 2A-F represent the results of the binding of the antibody produced in the small scale process of Example 1 (pH 6.5, 7.5 and 7.8 are results of full-scale purification).

TABLE 2

| PH of Capto | Aggregates | | KD2* | | | | | Ratio |
|---|---|---|---|---|---|---|---|---|
| adhere | (%) | KD* (M) | (M) | Kon (1/Ms) | kon2 (1/Ms) | kdis (1/s) | kdis2 (1/s) | KD1/KD2 |
| 5.5 | N.D | 1.40E−07 | 1.00E−12 | 6.91E+05 | 3.61E+04 | 9.70E−02 | 1.00E−07 | 65/35 |
| 6.1 | N.D | 1.84E−07 | 4.10E−08 | 6.38E+05 | 3.74E+04 | 1.17E−01 | 1.53E−03 | 50/50 |
| 6.5 | 0.8 | 3.06E−07 | 8.55E−08 | 4.77E+05 | 4.66E+04 | 1.47E−01 | 4.02E−03 | 80/20 |
| 7.5 | 0.3 | 3.69E−07 | 2.57E−07 | 4.31E+05 | 4.61E+04 | 1.52E−01 | 1.51E−02 | 60/40 |
| 7.8 | 0.2 | 3.76E−07 | 2.81E−07 | 4.11E+05 | 4.85E+04 | 1.54E−01 | 1.37E−02 | 50/50 |
| Xgeva ® | 0.9 | 3.28E−07 | 8.65E−08 | 4.33E+05 | 4.22E+04 | 1.42E−01 | 3.64E−03 | 80/20 |

*using a 2:1 heterogeneous ligand model results in 2 KDs

As may be observed from FIGS. 2A-F, a direct correlation exists between pH of the Capto adhere chromatography step to the sensorgram pattern obtained for binding of the mAb to FcγRIIa—increasing the pH decreases binding affinity. From binding affinity parameters presented Table 1 it may be observed that all parameters are gradually modified with increasing pH, but the most significant change is observed in Kdis2 in which a five orders of magnitude change is observed between pH 5.5 and 7.8. This decrease in dissociation rate is reflected in KD2. Another parameter which significantly changes upon modifying the pH of the Capto chromatography is the ratio between KD1 and KD2. The pH of Capto Adhere chromatography that yields a most similar sensorgram pattern and binding affinity parameters is pH 6.5.

The effect of pH of Capto adhere chromatography, at a range of 5.5-6.6, on binding to FcγRIIa has been tested with five different clones, grown in either flasks or bioreactors. Similar results were obtained with all clones after purification, regardless of the binding pattern observed in the harvests. The effect of pH 7.5 and 7.8 was analyzed in two different clones with similar results.

The effect of Capto adhere chromatography pH was also tested on binding to FcRn (not shown) In contrast to FcγRIIa, no effect of pH was obtained on binding affinity or relative binding to FcRn. FcRn is known from literature to be sensitive to aggregate content (1, 2, 3). Nevertheless, it was demonstrated that up to 5% aggregates in the sample does not interfere with the measurements performed using the Octet (1). These results may indicate that the modification in binding pattern observed in FcγRIIa affinity as a function of Capto adhere chromatography pH, does not result from aggregate content.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

(other references are cited throughout the document)
1. Bajardi-Taccioli A. et al. Effect of protein aggregates on characterization of FcRn binding of Fc-fusion therapeutics. Mol. Immunol. 67: 616-624 (2015).
2. Wu Q. et al. Development and applications of AlphaScreen-based FcRn binding assay to characterize monoclonal antibodies. J. Immunol. Methods: 420 31-37 (2015).
3. Schlothauer T. Analytical FcRn affinity chromatography for functional characterization of monoclonal antibodies. mAbs 5(4): 576-586 (2013).

1. A method of purifying a recombinant antibody having the complementarity determining regions (CDRs) of denosumab, the method comprising subjecting a preparation comprising said antibody to a mixed mode chromatography on a Capto™ Adhere column in a pH range of 6.2-7.4, thereby purifying the antibody.

2-14. (canceled)

15. The method of claim 1, wherein said pH range is 6.5-6.6.

16. The method of claim 1, wherein said subjecting comprises an equilibration buffer and a sample buffer.

17. The method of claim 1, further comprising subjecting said preparation to an affinity chromatography prior to said subjecting to said mixed mode chromatography.

18. The method of claim 1, further comprising subjecting said preparation to a cation exchange (CEX) chromatography prior to said subjecting to said mixed mode chromatography.

19. The method of claim 18, wherein said subjecting to said CEX chromatography is following said subjecting to said affinity chromatography.

20. The method of claim 1, wherein said affinity chromatography comprises a protein A resin.

21. The method of claim 20, wherein said protein A resin comprises mAbSelect SuRe™.

22. The method of claim 1, wherein said CEX chromatography comprises Eshmuno-S™ resin.

23. The method of claim 1, wherein each of said protein A resin and said Eshmuno-S™ resin are packed into a column.

24. The method of claim 1, further comprising a viral inactivation step prior to said subjecting to said mixed mode chromatography.

25. The method of claim 24, wherein said viral inactivation is prior to said CEX chromatography and following said affinity chromatography.

26. The method of claim 1, wherein said antibody is expressed in CHO cells.

27. A composition of matter comprising a purified recombinant antibody having the complementarity determining regions (CDRs) of denosumab obtainable by the method of claim 1, wherein said antibody has a binding affinity to FcγRIIa which is about the same as the binding of Xgeva® or Prolia®, as determined by BLI using the parameters of Example 1.

28. A method of qualifying a recombinant antibody batch comprising an antibody having the complementarity determining regions (CDRs) of denosumab, the method comprising determining a binding affinity of the antibody obtainable according to claim 1 to an Fc receptor, wherein a binding affinity is within the similarity range for similarity determined from 5 Xgeva® and Prolia® lots and inter assay variability as determined by BLI, is indicative that the antibody batch is a denosumab biosimilar.

29. The method of claim 28, wherein said Fc Receptor is FcγRIIa and said affinity is determined using the parameters to Example 2.
